(19) United States
(12) Patent Application Publication
(10) Pub. No.: US 2017/0181943 A1
(43) Pub. Date: Jun. 29, 2017

(54) METHODS AND COMPOSITIONS FOR ENHANCING AND EXTENDING THE COSMETIC EFFECTS OF NON-SURGICAL DERMAL INTERVENTIONS

(71) Applicant: Enhance Skin Products, Inc., Reno, NV (US)

(72) Inventors: Gary D. HACK, Columbia, MD (US); Samuel S. ASCULAI, Toronto (CA)

(73) Assignees: ENHANCE SKIN PRODUCTS, INC., Reno, NV (US); Gary D. HACK, Columbia, MD (US)

(21) Appl. No.: 15/301,426

(22) PCT Filed: Mar. 27, 2015
(86) PCT No.: PCT/US2015/023183

§ 371 Date: Oct. 3, 2016

Related U.S. Application Data
(60) Provisional application No. 61/974,559, filed on Apr. 3, 2014.

(51) Int. Cl. A61K 8/25; A61K 8/02; A61K 8/73; A61Q 19/08

(52) U.S. Cl. CPC A61K 8/25; A61Q 19/08; A61K 8/0241; A61K 8/735; A61K 2800/91

(57) ABSTRACT

The present invention relates to methods for enhancing, extending and accelerating the cosmetic benefits certain non-surgical dermal interventions, examples of which include treatment with aesthetic injectables such as botulinum toxin and dermal fillers, as well as intense pulsed light (IPL) treatments, particularly cosmetic treatments characterized by the injection of botulinum toxin compositions, such as Botox® and Dysport®, and/or dermal fillers, such as Restylane® and Juviderm®. More particularly, the present invention relates to the unexpected discovery that the combination of micron-sized particulate bioactive glass and medical grade hyaluronic acid yields a cosmetic composition that is capable of improving and prolonging the anti-wrinkle effects of such cosmetic therapies as well as to afford enhanced and improved healing and anti-microbial benefits at the site(s) of injection.

### PRIORITY

This application claims the benefit of U.S. Provisional Application Ser. No. 61/974,559 filed Apr. 3, 2014, the contents of which are incorporated by reference herein.

### FIELD OF THE INVENTION

The present invention relates to the use of a bioactive glass-containing topical skin-care product to enhance and extend the beneficial cosmetic effects of certain non-surgical dermal interventions, in particular the anti-wrinkle effects of cosmetic treatments associated with aesthetic injectables such as botulinum toxin and/or cosmetic dermal fillers.

### BACKGROUND TO THE INVENTION

Botox® is a drug made from a toxin produced by the bacterium Clostridium botulinum that is used medically to treat certain muscular conditions and cosmetically to remove wrinkles by temporarily paralyzing facial muscles. In cosmetic use, a tiny quantity of the toxin botulinum is injected into human tissue—mainly in the face. Botox® injections work by weakening or paralyzing certain muscles or by blocking certain nerve endings so as to prevent electrical signals from moving through muscles. The cosmetic effects include a wrinkle-free appearance.

Since its launch by Allergan in 1989, Botox® has become big business, with millions of people getting Botox® injections to combat wrinkles. Unfortunately, the downside is that the cosmetic benefits last an average of only three months, meaning regular trips to the doctor—and repeated costly injections—to maintain the wrinkle-free look. The life of dermal fillers, such as the hyaluronic acid gel fillers commercially available under the tradenames Restylane® and Juviderm®, is similarly limited, on the order of six to twelve months.

A prescription medication sold under the tradename Zytaze® (OcuSoft, Richmond, Tex.) has recently come on the market with claims to help Botox® take effect faster and make the wrinkle-smoothing effects of the cosmetic injections last 30% longer. In fact, a clinical trial of the pill, published in the Journal of Drugs in Dermatology, found that 92% of patients taking Zytaze® reported Botox® treatments lasted for several weeks longer than normal; in addition, Zytaze® was reported to help the injection to take effect more quickly. Administered in the form of an oral tablet, Zytaze® is essentially a vitamin supplement containing a high dose of zinc combined with an enzyme called phytase. Although the method of action remains unclear, Zytaze® appears to function by helping the body absorb zinc, a vitamin fundamental in repairing tissue and known to help injured patients recover from injuries such as burns.

While the efficacy and safety of Zytaze® remains to be confirmed, it is important to note that too much zinc (e.g., above 50 mg) can negatively alter the function of the immune system. Other medical problems that may result from an overdose of zinc include gastrointestinal symptoms of nausea, vomiting, diarrhea, and stomach pain as well as dizziness, kidney dysfunction, anemia, and decreased immune function. In a similar fashion, too much phytase on a regular basis may cause too much zinc absorption resulting in the previously mentioned negative side effects. Zinc may decrease the absorption of antibiotics such as tetracycline (Achromycin, Sumycin) and ciprofloxacin (Cipro). In addition, use of drugs such as aspirin; AZT (azidothymidine, zidovudine, Retrovir); captopril (Capoten); enalapril (Vasotec); estrogens (oral contraceptives and Premarin); penicillamine (Cuprimine); the thiazide class of diuretics including chlorothiazide (Diuril and others), chlorthalidone (Hygroton and others), hydrochlorothiazide (Esidrix, HCTZ, HydroDIURIL, Oretic, and others), metolazone (Mykrox and Zaroxolyn) can increase the loss of zinc from the body or interferes with its absorption. Zinc supplements may also be contraindicated for women who are pregnant, nursing or may become pregnant.

Given these negative side effects, there remains a need in the art for means, preferably a non-systemic topical means, to enhance and extend the cosmetic effects of non-surgical dermal interventions, examples of which include treatment with aesthetic injectables such as botulinum toxin and dermal fillers, as well as intense pulsed light (IPL) treatments, more particularly the anti-wrinkle effects associated with injectable botulinum toxin treatments such as Botox® as well as dermal fillers such as Restylane® and Juviderm®.

### SUMMARY OF THE INVENTION

Given the above described need in the art, it is a goal of the present invention to provide novel skin care products that enhance and/or extend the cosmetic effects associated with non-surgical dermal interventions such as injectable botulinum toxin treatments, examples of which include Botox® and Myobloc®, as well as injectable dermal fillers, examples of which include hyaluronic acid-based fillers Restylane® and Juviderm®. More particularly, the present invention is directed to novel skin care products that enhance and extend the anti-wrinkle effects of cosmetic treatment with botulinum toxin and dermal fillers. Hyaluronic Acid (HA) is a naturally occurring sugar polymer (glycosaminiglycan) of central biological importance. HA is present in every tissue of the body; in fact, three percent (3%) of the human body, by dry weight, is composed of HA. HA is found in particularly high concentrations in the skin, eyes, joints and between individual cells.

HA has many functions, including stimulating the tissue's water retention capabilities. A natural humectant, HA molecules can hold up to 400 times their own weight in water. This ability to bind and retain moisture allows HA to assist in the regulation of water balance, provide structural support to tissues and act as a lubricant. For example, HA in the eye serves to keep them round and in joints, as part of the synovial fluid, acts as a lubricant and shock absorber. However, 56% of the HA in the human body is found in the skin, where it helps retain moisture and structure.

Together with collagen and elastin, HA forms the cement that holds cells together.

HA, in conjunction with the connective tissue molecules collagen and elastin—known as ground substance—are the structural components of the epidermis and the dermis. These connective tissue molecules form a sponge-like matrix, the spaces of which are filled with HA and water. This HA-water complex ensures sufficient hydration so that normal skin function and cell turnover can occur.

Hyaluronan, hyaluronic acid, and its salts are known in the medical arts for topical applications of drugs (see e.g., U.S Pat. No. 6,218,373). They are also known in the cosmetic arts for its greater moisturizing and nourishing properties as well as its benefits to collagen. Studies have shown (see, e.g., Stern, R J, 2006, Hyaluronan: Key to Skin Moisture, 246-277, In: Dry Skin and Moisturizers, Loden, M and HI Maibach Eds.) that fragmentation of the HA polymer generates size-specific pieces, or oligomers, with widely differing biological activities. However, it is difficult to synthesize HA free of contaminating glycoprotein, lipids and other tissue material in the laboratory setting. In spite of these drawbacks, many cosmetic and cosmeceutical manufacturers continue to incorporate “cosmetic grade” HA into their products and claim their beneficial effects for their products. However, HA that is impure and ill-defined as to molecular size, pH and biological activities can be less effective.

Scientists have worked to identify and isolate HA molecules of a size and purity that will result in maximum hydration, optimal intradermal delivery, systematic targeting and safety. An HA oligomer of extremely high purity that provides the hydrating, delivery and targeting characteristics not found in “cosmetic grade” HA is described by Dr. Samuel Asculai in U.S. Pat. No. 8,758,819, the contents of which are incorporated by reference herein. In the '819 patent, Dr. Asculai describes novel compositions, particularly acidic compositions having a pH of 5.2 to 5.5, composed of “medical grade” HA, e.g., HA on the order of about 700 KiloDaltons, formulated with bioactive/biocompatible microparticulates such as bioactive glass or bioactive ceramics. Dr. Asculai discovered that such compositions are anti-inflammatory, hypoallergenic, non-irritating, fragrance free, non-comedogenic and oil-free and thus are ideal for cosmetic topical application. Dr. Asculai describes such compositions as able to reduce and treat skin roughness and inflammation, enhance and accelerate healing, inhibit infection, and enhance skin tone and moisture as well as diminish dermal aging such as wrinkle formation and improve the water retaining ability of normal skin. Accordingly, he finds such compositions are uniquely suited to the treatment of skin after non-surgical ablative skin resurfacing procedures such as chemical peels, microdermabrasion, laser peels and the like. As such, the compositions described in U.S. Pat. No. 8,758,819 are now a primary component of the Visible Youth™ skin care line commercially available through Enhance Skin Care Products (Denver, Colo.), examples of which include the Visible Youth Revitalizing Cleanser™ Visible Youth Revitalizing Lotion™; Visible Youth Revitalizing Moisturizer™; Visible Youth Eye Zone Gel™; Visible Youth Healing Complex™; Visible Youth Healing Complex +3% lidocaine™ (a prescription-only product).

It is an object of the present application to combine a hyaluronan, hyaluronic acid, and/or biological active salt, homologue, analogue, derivative, complex, ester, fragment, or subunit thereof, particularly the purified medical grade HA products described in U.S. Pat. No. 8,758,819 referenced above, with small particle bioactive glasses, with and without the addition of silver ions, such as described in published U.S. Pat. No 6,482,444 and U.S. Pat. No. 8,722,080, also incorporated herein by reference, to form a topical serum that may be applied to the skin to enhance and extend the cosmetic effects of non-surgical dermal interventions, examples of which include botulinum toxin injections, dermal fillers, and intense pulsed light (IPL) treatments, especially the anti-wrinkle benefits derived from botulinum toxin injections and Botox® and dermal filler treatments conventionally applied to reduce facial wrinkles.

Accordingly, in one aspect, the present invention provides a method of enhancing and extending the wrinkle reduction associated with Botox® or its generic equivalent, said method comprising the step of repeatedly administering an effective amount of a cosmetic composition to a patient who has recently received one or more cosmetic injections of botulinum toxin, wherein said cosmetic composition comprises at least one bioactive/biocompatible microparticulate in combination with an intradermal delivery vehicle selected from the group consisting of hyaluronans, hyaluronic acid and/or salts thereof and/or homologues, analogues, derivatives, complexes, esters, fragments and subunits of hyaluronic acid in an amount sufficient to facilitate deposition and penetration of said bioactive microparticulates and/or its constituent components through tissue at a site to be treated. Preferably the cosmetic composition is acidic, having a pH on the order of 5.2 to 5.5 such as described in the '819 patent discussed above and incorporated by reference herein.

In addition, minor adverse events such as bruising, bleeding, pain, redness, discomfort, infection, and/or swelling can occur where the injections are given. It is a further objective of the present invention to utilize the herein described cosmetic composition to minimize or reduce the extent of these commonly occurring post-injection complications.

Preferably, the cosmetic composition is formulated for topical use, to be applied daily or multiple times a day and is targeted to the one or more injection sites. The cosmetic composition may be in aqueous or non-aqueous form, particularly in the form of a lotion, cream, gel or combination thereof. Such cosmetic compositions for the treatment of skin may, in addition to the above contains normally found cosmetic excipients such as oils, gums, glycerin, preservatives, water, etc.

The bioactive microparticulates may be bioactive glasses, bioactive ceramics, bioactive minerals or composites of these. Preferably, the microparticulate is a melt-derived bioactive glass. However, compositions prepared using a sol-gel method such as described in U.S. Pat. No. 6,482,444 referenced above and incorporated by reference herein are also contemplated. The present invention also contemplates the optional inclusion of additional therapeutic agents such as antibiotics and/or antimicrobials, e.g., a silver salt selected from among silver oxide, silver nitrate, silver acetate, silver bromide, and silver chloride.

Preferably, the intradermal delivery vehicle is hyaluronic acid. More preferably, the hyaluronic acid is of medical grade and has an average molecular weight of about 700 kiloDaltons.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims.

### DETAILED DESCRIPTION

As noted, the present invention is directed to a topical serum comprised of purified medical grade HA products combined with small particle bioactive glasses, melt-derived or sol-gel bioactive glass, with and without the addition of silver ions that may be applied to the skin to enhance and extend the benefits derived from Botox treatments to reduce facial wrinkles.

Bioactive glasses are known mainly as bone grafting materials. Their beneficial biological activity and high level of biocompatibility are well documented. Recent demonstrations evidenced that finely grained powders of bioactive glasses have substantial anti-microbial, anti-inflammatory and mineralizing properties.

However, when formulated in an aqueous solution, the pH of the solution tends to rapidly rise to an unacceptably high alkaline level, e.g., on the order of 11 or 12. As such, aqueous formulations of bioactive glass alone are unsuited to cosmetic applications. However, as discussed in the '819 patent referenced above and incorporated by reference herein, when bioactive glassis formulated with a medical grade HA in an aqueous environment, the pH remains surprisingly stable, buffered to a slightly acidic pH that can be beneficial for the skin. As discovered herein, such a pH-stabilized composition finds novel and unexpected utility in extending and enhancing the cosmetic effects of non-surgical dermal interventions, particularly cosmetic therapies that involve the application of aesthetic injectables such as botulinum toxin and/or dermal fillers.

### Definitions

All patents and publications referred to herein are incorporated by reference to the same extent as if each individual publication was specifically and individually indicated as having been incorporated by reference in its entirety.

Unless otherwise defined, all technical and scientific terms used herein have the same meaning as commonly understood by one of ordinary skill in the art to which this invention belongs, in accordance with conventional usage. To that end, definitions of common terms in molecular biology may be found, for example, in Benjamin Levin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references. However, in case of conflict, the present specification, including definitions, will control. Accordingly, in the context of the present invention, the following definitions apply:

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, “treat” and all its forms and tenses (including, for example, treating, treated, and treatment) can refer to therapeutic or prophylactic treatment and encompasses any observable or measurable improvement in a sign or symptom of a condition. In the context of cosmetic treatment, the primary goal of the present invention is to provide a prolonged elimination or reduction in the appearance of fine lines and wrinkles on the face, particularly in the areas around the eyes and lips and on the forehead, that arise from botulinum toxin injections, as well as injections of dermal fillers such as Restylane® and Juviderm®. Secondary improvements may include a reduction in inflammation or erythema, improved skin texture and elasticity, a reduction in pore size, and hydration of the skin in conditions such as rosacea, acne, eczema, dermatitis, bed-sores, burns, scars, and diabetic ulcers.

In the context of the present invention, the terms “subject” and “patient are interchangeably used to refer to humans undergoing the described therapy.

As used herein, the term “average particle size” in general means that some particles will be smaller and some particles will be bigger than the size specified. For purposes of this application and by way of example, where a composition contains bioactive glass particles of an average particle size of less than about 10 microns, typically 90-95% of the particles will be less than about 20 microns. Where the composition contains bioactive glass particles of an average particle size of less than about 5 microns, typically 90-95% of the particles will be less than about 15 microns. Where the composition contains bioactive glass particles of an average particle size of less than about 2 microns, typically 90-95% of the particles will be less than about 6 microns.

In the context of the present invention, the terms “bioactive glass” or “biologically active glass” are used interchangeably to refer to an inorganic glass material having an oxide of silicon as its major component and that is capable of bonding with growing tissue when reacted with physiological fluids. Bioactive glass interacts with the body's tissues to stimulate cell growth and provide vital anti-bacterial, structural and/or regenerative properties. By way of example, a bioactive glass in accordance with the invention is a glass composition that will form a layer of hydroxycarbonate apatite in vitro when placed in a simulated body fluid. A bioactive glass suitable for use in connection with the present invention must be non-toxic, resorbable and biocompatible such that it does not trigger an overwhelmingly adverse immune response in and on the body, such as in the skin and epidermis. Bioglass materials may be fabricated by a melt-derived or sol-gel process, have a high silicon and a low nitrate content, can be provided as a solid or a powder and can easily be combined with gels and sprays. Illustrative examples, are described in detail below and include TheraGlass® compositions (TheraGlass Limited, London, UK), with and without the addition of silver.

In the context of the present invention, the term “botox” refers to a purified form of botulinum, a neurotoxin produced by the bacterium Clostridium botulinum that causes botulism, that is injected in minute amounts especially to treat muscle spasms and relax facial muscles in order to reduce wrinkles. Commercially available botox products include:

Botox® by Allergan;

Myobloc® by Solstice Neurosciences;

Dysport® by Ipsen, Ltd; and

Xeomin® by Merz.

Although all four products are made from the Clostridium botulinum bacteria, the four products are isolated from different strains of bacteria, have different molecular structures, and attach to different parts of the nerve. For example, whereas Botox, Xeomin, and Dysport are Botulinum toxin type A, Myobloc is type B. Critically, the utility of the present invention is not restricted to any particular form of botox.

In the context of the present invention, the term “dermal filler” refers to a product that is injected or placed into the dermis; in the context of cosmetics, injectable dermal fillers are often used as “volumizers”, to enhance facial shaping, plump lips and reduce the sight of fine lines and wrinkles, as well as reduce the appearance of deep wrinkles and folds. The present invention finds utility in connection with a wide array of dermal fillers including:

hyaluronic acid fillers such as Restylane® and Juviderm®;

synthetic fillers such as calcium hydroxylapatite available under the tradename Radiesse® and synthetic poly-L-lactic acid available under the tradename Scupltra®;

collagen fillers such as Cosmoplast® and Cosmoderm®; and

autologous fillers, typically fat tissue.

### Elements of the Present Invention

As noted above, the present invention combines hyaluronic acid, particularly purified medical grade HA products described in published U.S. Pat. No. 8,758,819 incorporated by reference herein, with small particle bioactive glasses, with and without the addition of silver ions, such as described in U.S. Pat. No 6,482,444 and U.S. Pat. No. 8,722,080, both of which are incorporated herein by reference, to form a topical serum that may be applied to the skin to enhance and extend the benefits derived from Botox treatments to reduce facial wrinkles.

### Hyaluronic Acid

Hyaluronic acid has been taught and used previously for topical applications of drug. See for example the following U.S. patents, incorporated by reference herein:

TABLE 1
HYALURONIC ACID PATENTS

| | |
|---|---|
| 8,758,819 | Cosmetic compositions for the treatment of skin and methods |
| | thereof |
| 6,218,373 | Formulations containing hyaluronic acid |
| 6,194,392 | Treatment of conditions and disease |
| 6,159,955 | Use of hyaluronic acid and a NSAID for the manufacture |
| | of a medicament for the treatment of mucosal diseases |
| 6,147,059 | Formulations containing hyaluronic acid |
| 6,140,312 | Formulations containing hyaluronic acid |
| 6,136,793 | Formulations containing hyaluronic acid |
| 6,114,314 | Formulations containing hyaluronic acid |
| 6,103,704 | Therapeutic methods using hyaluronic acid |
| 6,087,344 | Formulations containing hyaluronic acid |
| 6,069,135 | Use of hyaluronic acid or its derivatives to enhance |
| | delivery of therapeutic agents |
| 6,048,844 | Treatment of conditions and disease |
| 6,017,900 | Topical composition containing hyaluronic acid and NSAIDS |
| 5,990,096 | Formulations containing hyaluronic acid |
| 5,985,850 | Compositions comprising hyaluronic acid and drugs |
| 5,977,088 | Formulations containing hyaluronic acid |
| 5,972,906 | Treatment of mucous membrane disease, trauma or |
| | condition and for the relief of pain thereof |
| 5,962,433 | Topical composition containing hyaluronic acid and NSAIDS |
| 5,942,498 | Formulations containing hyaluronic acid |
| 5,932,560 | Treatment of conditions and disease |
| 5,929,048 | Treatment of conditions and disease |
| 5,914,322 | Treatment of disease and conditions |
| 5,910,489 | Topical composition containing hyaluronic acid and NSAIDS |
| 5,852,002 | Treatment of conditions and disease |
| 5,830,882 | Compositions containing a form of hyaluronic acid |
| | and a medicinal agent for treating acne in mammals and |
| | methods for administration of such composition |
| 5,827,834 | Method of using hyaluronic acid or its pharmaceutically |
| | acceptable salts for the treatment of disease |
| 5,824,658 | Topical composition containing hyaluronic acid and NSAIDS |
| 5,792,753 | Compositions comprising hyaluronic acid and |
| | prostaglandin-synthesis-inhibiting drugs |
| 5,639,738 | Treatment of basal cell carcinoma and actinic |
| | keratosis employing hyaluronic acid and NSAIDs |

The present invention contemplates the use of hyaluronans, hyaluronic acid and/or salts thereof and/or homologues, analogues, derivatives, complexes, esters, fragments and subunits of hyaluronic acid in an amount sufficient to facilitate deposition and penetration of the below-noted bioactive microparticulates through tissue at a site to be treated. Preferably, the intradermal delivery is hyaluronic acid. More preferably, the hyaluronic acid is of medical grade, has an average molecular weight of about 700 kiloDaltons.

### Bioglass

The present invention contemplates the use of a commercially available bioactive microparticulate commonly referred to in the art as “Bioglass” that is composed of SiO₂, Na₂O, CaO, and P₂O₅ in specific proportions. The proportions differ from the traditional soda-lime glasses in low amount of silica (less than 60 mol. %), high amount of sodium and calcium, and high calcium/phosphorus ratio. High ratio of calcium to phosphorus promotes formation of apatite crystals, wherein calcium and silica ions can act as crystallization nuclei. Bioglasses are generally divided to two categories:

Class A bioglasses bind with both soft tissues and bone to give rise to an HCA layer that forms within several hours.

Class B bioglasses bind with bone to give rise to an HCA layer takes one to several days to form.

In the context of the present invention, Class A Bioglasses are more preferred.

A detailed discussion of the various Bioglass compositions may be found at http://en.wikipedia.org/wiki/Bioglass#cite_note-biomat-0, the contents of which are incorporated by reference herein.

Particulate “Bioglass” materials suitable for us in the context of the present invention typically have the following composition by weight percentage:

TABLE 2

| Material | Weight Percent |
|---|---|
| SiO₂ | 40-90%, more preferably 40-60%, more preferably |
| | 40-50% |
| CaO | 6-50%, more preferably 10-30%, more preferably |
| | 20-25% |
| Na₂O | 0 to 40%, more preferably 10-35%, more preferably |
| | 20-25% |
| P₂O₅ | 0-12%, more preferably 2-8%, more preferably |
| | 4-6% |

Higher CaO contents provide larger pore volumes and the onset of hydroxycarbonate apatite (HCA) deposition is accelerated. Bioglasses with higher SiO₂ contents tend to have larger surface areas and exhibit higher growth rates of formation of an HCA layer.

In certain embodiments, CaO can be replaced with MgO and some Na₂O with K₂O. CaO can also be replaced with CaF₂, though this tends to modify the dissolution rate of the glass. B₂O₃ or Al₂O₃ may be added for easier material processing.

TABLE 3

| Material | Weight Percent | |
|---|---|---|
| MgO | 0-5% | (optional) |
| K₂O | 0-8% | (optional) |
| CaF₂ | 0-25% | (optional) |
| B₂O₃ | 0-10% | (optional) |
| Al₂O₃ | 0-1.5% | (optional) |

A number of species of bioactive glass are commercially available, examples are described in Table 4 below.

TABLE 4
Composition (mol. %) of bioactive glasses

| Designation | SiO₂ | CaO | P₂O₅ |
|---|---|---|---|
| 49S | 50 | 46 | 4 |
| 54S | 55 | 41 | 4 |
| 58S | 60 | 36 | 4 |
| 63S | 65 | 31 | 4 |
| 68S | 70 | 26- | 4 |
| 72S | 75 | 21 | 4 |
| 77S | 80 | 16 | 4 |
| 86S | 90 | 6 | 4 |

One particularly preferred species of bioactive glass is sold under the name “Bioglass 45S5” and has the following characteristics:

TABLE 5

| Material | Weight Percent |
|---|---|
| SiO₂ | 45% |
| CaO | 24.5% |
| Na₂O | 24.5% |
| P₂O₅ | 6% |

A similar bioactive glass species is commercially available through Schott North America (Southbridge, Mass.) under the tradename “Vitryxx®”. Vitryxx® bioactive glass (CAS: 65997-17-3) has the following composition:

TABLE 6

| Material | Weight Percent |
|---|---|
| SiO₂ | 45 +/− 5% |
| CaO | 24.5 +/− 3% |
| Na₂O | 24.5 +/− 3% |
| P₂O₅ | 6 +/− 2% |

### Theraglass®

In the context of the present invention, the bioactive glass may be fabricated by the melt-derived process described in U.S. Pat. No. 8,722,080 or the sol-gel process described in U.S. Pat. No. 6,483,444. An illustrative bioactive glass species suitable for use in the context of the present invention is commercially available under the trade name Theraglass® and also includes Silver Theraglass® (Theraglass Limited, London, UK). Unlike the first generation of bioactive glasses described in U.S. Pat. No. 8,722,080, which are made using a very high temperature “melt” process, Theraglass® materials are made by a unique sol-gel process. Sol-gel glasses (like the Theraglass® line of products) can be formulated into, for example, ointments, creams, pastes, gels, and sprays. The regenerative nature of this material applies itself to the repair of teeth, bones, and skin. It is classified as a medical device ingredient and is able to provide a long lasting and strong antimicrobial effect and a barrier to infection. The sol-gel process and resultant bioactive material gives a significant advantage in terms of versatility and corresponding uses. Theraglass® takes less energy and is less expensive to make. It is more uniform in consistency and can be controlled and adapted to the nano-level, thereby improving its performance for specific applications. For remineralization (HCA formation) it has been shown that 70% SiO₂ is optimal.

The morphology of the gel surface layer is a key component of the bioactive response. Studies on bioactive glasses derived from sol-gel processing have shown that such materials may contain significantly higher concentrations of SiO2 than traditional melt-derived bioactive glasses (e.g., on the order of 65% and higher) yet unexpectedly still maintain bioactivity (i.e., the ability to form a mineralized hydroxyapatite layer on the surface). While not wishing to be bound by theory, it has been proposed that the inherent porosity enables the retention, and indeed enhancement, of bioactivity.

The Theraglass® material may optionally include an effective amount of an antibacterial silver salt, examples of which include silver oxide, silver nitrate, silver acetate silver chloride, silver bromide. In the context of the present invention, the term “effective amount” refers to an amount effective to reduce the amount of bacteria in an area proximate to where the bioactive glass is present. This amount would be expected to vary depending on a variety of factors, including the type of bacteria, the type of bacterial concentration, the type of media, and the intended use. The bioactive glass compositions can be adjusted to include a variety of concentrations of silver ions and those skilled in the art can readily determine an appropriate antibacterial amount of silver to use.

In the context of the present invention, silver oxide (Ag₂O) is the preferred antibacterial agent; such an embodiment is commercially available under the trade name Silver Theraglass®. Recent studies have shown that the introduction of silver oxide into bioactive glass compositions can minimize the risk of microbial contamination through the potential antimicrobial activity of the leaching Ag+ ions. More particularly, it has been shown that a bioactive glass composition doped with silver oxide elicits rapid bactericidal reaction. As noted above, the bioactive glasses produced by the sol-gel process are uniquely able to maintain bioactivity even with high concentrations of silicon oxide. Additionally, the sol-gel process permits the tailoring of the textural characteristics of the matrix, which, in turn permits the controlled time-release of powerful antimicrobials like ionic silver. Thus, sol-gel process yields bioactive glass materials having a level and duration of antimicrobial and therapeutic activity that simply cannot be achieved with the melt-derived process.

Sol-gel produced Theraglass® has 2 or 3 components compared to the 4 components in melt-derived glasses. It has a strong ability to allow incorporation of other materials, and has a superior bioactivity level due to its greater surface area. The surface area of the sol-gel glasses is two orders of magnitude (i.e. 100 times) than that of the melt-derived glasses. An interconnected network of mesopores (2-50 nm) is responsible for the high surface area, which provides enhanced bioactivity. This increases effectiveness, e.g. only very small amount of material is required to achieve the therapeutic result. In addition, it is more porous, enabling other materials, proteins, or drugs (such as silver and zinc) to be incorporated into its structure and delivered in a time-release fashion. The ion release from sol-gel glasses such as Theraglass® occurs when it comes in contact with body fluids or is formulated in an aqueous solution. This leads to the formation of hydroxyl carbonate layer (HCA) on protein-containing surfaces such as teeth and mucosal tissues. In addition, it stimulates healing of dermal and osseous tissue. It has been shown to be safe, and if it enters the body it is resorbed and harmlessly excreted.

Silver Theraglass® has very strong antimicrobial properties arising through the action of silver, attributed to the significant surface area of the material, combined with the healing properties of the bioactive glass. The silver is released over time thereby providing lasting protection. See Saravanapavan P. et al, “Binary Cao-Sio(2) Gel-Glasses For Biomedical Applications” Biomed Mater Eng. (2004) Vol. 14, No. 4, pp. 467-86.

Particulate “Theraglass” materials suitable for us in the context of the present invention typically contain at least 50%, more preferably at least 60%, even more preferably at least 70% silicon oxide (SiO₂). Components such as silver and/or zinc, as well as CaO, MgO, CaF₂, Na₂O, K₂O, P₂O₅, B₂O₃, Al₂O₃, may also be included, within ranges similar to those utilized by the melt-derived bioglass materials. One of skill in the art may readily determine the optimal weight percentages of these additional ingredients.

### Bioactive Compositions of the Present Invention:

As noted above, the present invention relates to the unexpected discovery that an acidic formulation of micron-sized particulates of bioactive glass with a medical grade hyaluronic acid-based products yields a topical composition that is capable of enhancing and extending the beneficial cosmetic effects of non-surgical dermal interventions, examples of which include aesthetic injectables such as botulinum toxin and dermal fillers as well as intense pulsed light (IPL) treatments, more particularly the anti-wrinkle effects of botox and dermal filler injections. While not being bound to any particular theory or mechanism, it is believed that the high surface area and reactivity of particulate bioactive glass provides for a release of sodium (a component found in many bioactive glasses) as well as the precipitation of calcium and phosphorus naturally present in the dermal tissues, which causes the rapid formation of a calcium and phosphate layer that, may incorporate collagen, fibrin and fibronectin to stabilize the skin.

The bioactive glass/HA compositions of the present invention are most effective when sized to enter the pores of or be absorbed by the skin. A typical pore has a diameter on the order of 50 microns. Accordingly, the preferred particle size for the bioactive glass less than 50 microns, preferably less than 25 microns, more preferably less than 10 microns can also be used. Particles of such a small size range generally provide for the advantages of the present invention without any undesirable immune response.

As discussed in greater detail in the next section, the active agents, bioactive glass and hyaluronic acid, can be combined in any pharmaceutically acceptable carrier to facilitate application to the skin. The resulting formulations may take the form of a solid, liquid, paste or gel. For example, the compositions of the present invention may be formulated with an ointment, white petrolatum, mineral oil, glycerin, and other vehicle known to those of ordinary skill in the art.

### Administration and Dosage:

A cosmetic composition of the present invention, formulated to enhance, extend and/or accelerate the beneficial cosmetic effects of a non-surgical dermal intervention involving aesthetic injectables such as Botulinum toxin and dermal fillers, is preferably directly or topically administered and thus preferably takes the form of a topical preparation, for example, a lotion, cream or gel, derived by admixing the active agents (i.e., the microparticulate bioactive glass+HA delivery vehicle) with a suitable base material that is inactive against the active agent(s). Likewise, the active agents can be mixed with cosmetically acceptable carriers or media, examples of which include sterilized water, physiological saline, plant-oils, emulsifiers, suspending agents, lubricants surfactants, stabilizers, vehicles, preservatives, binders, adhesives, and such. The relative amounts of active ingredient contained in such a preparation makes a suitable dosage within the indicated range acquirable. As noted above, the active agents may be utilized in their native state or in the form of a pharmaceutically acceptable salt thereof.

The cosmetic compositions of the present invention are to be administered to a subject who has previously received one or more cosmetic procedures, particularly non-surgical dermal interventions, more particularly a treatment associated with an aesthetic injectable such as botulinum toxin and dermal fillers. Accordingly, the cosmetic compositions are preferably administered to the site of a prior injection. However, the target site may be extended to include facial regions of interest, such as the area around the mouth and eyes as well as the forehead.

In order to maximize effectiveness, the cosmetic compositions of the present invention are preferably administered daily, more preferably multiple times a day, e.g., twice a day, once in the morning and once at night.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

### Example 1

### Impact of HA on Formulation pH

It has been reported that suspension supernatants of micrometric (100 um) bioactive glass attained a pH of 8.3 after 10 minutes, whereas a pH of 11.7 was similarly achieved with nanometric (20-60 nm) bioactive glass. Thus, it has been suggested that by decreasing the size of the bioactive glass particles, a pH elevation of over three units can be achieved, due to the increase of the specific surface area and the release of more alkaline species.

The in vitro behavior of Bioglass 45S5 was investigated herein. The “45S5” name signifies glass with 45 wt. % of SiO₂ and 5:1 ratio of CaO to P₂O₅. The key composition features of Bioglass is that it contains less than 60 mol % SiO₂, high Na₂O and CaO contents, high CaO/P₂O₅ ratio, which makes Bioglass highly reactive to aqueous medium and bioactive. Bioglass 45S5 with five particle size distributions was studied by measuring the in situ pH after immersion in an aqueous physiologic saline. As discussed in greater detail below, an immediate and sustained elevation in pH was observed regardless of particle size.

TABLE 7

| | | pH | |
|---|---|---|---|
| | | (reached within 30 | |
| Average Particle Size | | seconds) | Observation |
| 100 | um | 11 | Higher than the pH of 8.3 |
| | | | observed by others after 10 |
| | | | minutes. |
| 16 | um | 12.7 | Higher than nanometric |
| | | | pH. |
| 5 | um | 13.02 | Higher than calcium |
| | | | hydroxide pH. |
| 2 | um | 12.6 | Higher than nanometric |
| | | | pH. |

It has been suggested that by decreasing the size of the Bioglass particles, a significant increase in pH can be achieved, due to the increase of the specific surface area and the release of more alkaline species. Accordingly, the ability of aqueous Bioglass 45S5 suspensions, utilizing an array of particle sizes, to raise the local pH was assayed via microvolt measurements. Different particle sizes of Bioglass 45S5 were used: 300-500 um, 100-300 um, <100 um, 16 um, 5 um. Time vs. maximum pH was measured, as well as plateau duration. Suspension supernatants (1:10 wt/wt) of micrometric (100 um) BG evidenced a pH of 8.3 after 10 minutes, whereas a pH of 11.7 was similarly achieved with nanometric (20-60 nm) BG. Unexpectedly, it was discovered that micron-sized particles of Bioglass 45S5 produced an immediate and sustained elevation in pH. The pH change has been previously shown to vary depending on the surface area to volume ratio, sample dosage. By studying the pH changes in the solution, a fast and simple determination of the in vitro behavior of the glass can be determined.

While elevated pH is preferred in certain utilities, it is most definitely not preferred in the context of skin treatment. To address the pH problem, hyaluronic acid is added to buffer the pH to slightly acidic, a condition that is preferred for skin therapy.

Illustrative examples of cosmetic formulations suitable for use in the context of the present invention are as follows:

TABLE 8

| | Parameter | Example 1 | Example 2 |
|---|---|---|---|
| Purified water | 90 to 98% | 96.6% | 97.1% |
| Hyaluronic Acid or Sodium | | | |
| Hyaluronate | 0.1 to 1.0% | 0.5% | 0.5% |
| Microparticulate bioactive | | | |
| glass (e.g., Calcium Sodium | 0.5 to 2.0% | | |
| Phosphosilicate) | | 0.95% | 0.95% |
| Citric Acid | 0.5 to 2.0% | 0.9% | 0.9% |
| Mica | 0.01 to 0.1% | 0.05% | 0.05% |
| Phenoxyethanol (optional) | 0.1 to 1.0% | 0.8% | |
| Preservative | 0.1 to 1.0% | 0.2% | 0.5% |
| | | | (Caprylyl |
| | | | Glycol) |
| | 100.0% | 100.0% | 100.0% |

### Example 2

### Enhancing Cosmetic Effects of Non-Surgical Dermal Interventions

Cosmetic compositions such as described in Examples 1 and 2 are expected to fundamentally improve, maintain, or enhance the protective and maintenance performance of normal skin after rejuvenation procedures. To that end, they find utility in reducing inflammation of skin tissue, enhance and accelerate healing of skin tissue (including wounds), inhibit infection of skin tissue, enhance skin tone, and moisturize the skin and have been shown to improve skin texture, evenness, and luminescence in test subjects. See FIGS. 1-3 of U.S. Pat. No. 8,758,819, incorporated by reference herein. In addition, they have been found to address persistent rosacea and eczema (data not shown).

Herein, in accordance with the present invention, such compositions are demonstrated to significantly enhance and extend the cosmetic effects of certain non-surgical dermal interventions, more particularly to improve and augment the anti-wrinkle effects of cosmetic treatments associated with injection of botulinum toxin and injectable dermal fillers.

To quantity such effects, ten patients are randomly selected from a group of patients receiving facial enhancement treatments at a clinic that offers Botulinum Toxin and Dermal Filler treatments for the reduction of facial rhytides (wrinkles). In addition to the facial injections, the selected patients also receive a topical facial cream containing hyaluronic acid and compositions of micro-particulate bioactive glasses such as described in Table 8 above. The facial cream is applied daily (single dose in the evening) at home to the treated facial regions, usually including the forehead, nasiolabial folds, lateral canthus of the eyes (crow's feet), around the lips, and the Glabellar Complex (area between the eyes).

The patients are carefully followed for at least 7 months following the injections so as to measure the impact of the facial cream on the de-wrinkling effects of the BT injections. To that end, patients keep a personal log and return to the treating office initially one week after the injections and then every 6 weeks thereafter. In addition to examining the patient's facial appearance at every follow-up appointment, photography is utilized to document each case at the various time intervals.

The de-wrinkling effects botulinum toxin (BT) injections last on average three to four months and the initial results can take several days or longer to appear after the injections. In accordance with the present invention, treatment with a facial cream such as described in Table 8 above, composed of micron-sized particulate bioactive glass formulated with a medical grade hyaluronic acid component, is expected to significantly extend, accelerate, and/or enhance the de-wrinkling effects of the BT injections.

### INDUSTRIAL APPLICABILITY

The present invention is directed to the use of micron-sized particulate bioactive glass compositions in combination with medical grade hyaluronic acid products to extend and enhance the beneficial cosmetic effects of certain non-surgical dermal interventions, examples of which include aesthetic injectables such as botulinum toxin and dermal fillers, as well as intense pulsed light (IPL) treatments, more particularly to extend, accelerate, and/or enhance anti-wrinkle effects of botulinum toxin injections and/or the injection of dermal fillers such as Restylane® and Juviderm®.

With respect to the above description, it is to be understood by the person skilled in the art that the dimensional relationships for the parts of the invention, include obvious variations of materials, form, function and manner of operation, assembly and use, and therefrom all equivalent relationships to those described in the specification are intended to be encompassed by the present invention.

Therefore, the foregoing is considered as illustrative only of the principles of the invention. Further, since numerous modifications and changes will readily occur to those skilled in the art, the invention is not limited to the exact construction and operation shown and described, and accordingly, all suitable modifications and equivalents that may be resorted to, fall within the scope of the invention.

What is claimed:

1. A method of enhancing, extending and/or accelerating the anti-wrinkle effects associated with an aesthetic cosmetic injectable selected from among botulinum toxin and dermal fillers, said method comprising the step of repeatedly administering an effective amount of a topical cosmetic composition to a patient who has recently received one or more of said cosmetic injections, wherein said cosmetic composition comprises at least one bioactive/biocompatible microparticulate in combination with an intradermal delivery vehicle selected from the group consisting of hyaluronans, hyaluronic acid and/or salts thereof and/or homologues, analogues, derivatives, complexes, esters, fragments and subunits of hyaluronic acid in an amount sufficient to facilitate deposition and penetration of said bioactive microparticulates through tissue at a site to be treated.

2. The method of claim 1 wherein said cosmetic composition is applied daily or multiple times a day.

3. The method of claim 1, wherein said cosmetic composition is applied to the one or more injection sites.

4. The method of claim 1, wherein said cosmetic composition is in aqueous form.

5. The method of claim 1, wherein said cosmetic composition comprises a lotion, cream, gel or combination thereof.

6. The method of claim 1, wherein said cosmetic composition further comprises one or more cosmetic excipients selected from the group consisting of oils, gums, glycerin, preservatives, and water.

7. The method of claim 1 wherein said bioactive/biocompatible microparticulate is a sol-gel derived or melt-derived bioactive glass.

8. The method of claim 7, wherein said bioactive/biocompatible microparticulate is a sol-gel derived bioactive glass comprising at least 65% silicon oxide.

9. The method of claim 7, wherein said bioactive/biocompatible microparticulate is a melt-derived bioactive glass comprising 40-50% silicon oxide.

10. The method of claim 1, wherein said bioactive/biocompatible microparticulate further comprises silver or zinc ions.

11. The method of claim 10, wherein said bioactive/biocompatible microparticulate comprises 0.1 to 15% by weight silver oxide.

12. The method of claim 1, wherein said intradermal delivery is hyaluronic acid.

13. The method of claim 12, wherein said hyaluronic acid is a medical grade product having an average molecular weight of about 700 kiloDaltons.

14. The method of claim 1, wherein the cosmetic composition is acidic.

15. The method of claim 14, wherein the pH of the cosmetic composition ranges from 5.2 to 5.5.

16. The method of claim 1, wherein said method further results in enhanced and/or accelerated healing of the skin tissue at the site of said cosmetic injections.

17. The method of claim 1, wherein said method further minimizes or reduces the extent of one or more commonly occurring adverse side effects associated with cosmetic injection, said side effects selected from the group consisting of discomfort, infection bruising, bleeding, pain, redness, and swelling at the injection site.

18. The method of claim 1, wherein said bioactive/biocompatible microparticulate has an average particle size of less than 10 microns.

19. The method of claim 1, wherein said bioactive/biocompatible microparticulate has an average particle size of less than 5 microns.

20. The method of claim 1, wherein said bioactive/biocompatible microparticulate has an average particle size of less than 2 microns.
